(19) United States
(12) Patent Application Publication
(10) Pub. No.: US 2024/0033328 A1
(43) Pub. Date: Feb. 1, 2024

(54) ACTH IN THE TREATMENT OF AN ADRENOCORTICOTROPIC HORMONE RESPONSIVE PEDIATRIC DISORDER

(71) Applicant: Amzell B.V., Hoofddorp (NL)

(72) Inventors: Dario Norberto Ramon CARRARA, Biel-Benken (CH); Laetitia Anne Christine HENRY-DELPY, Sierentz (FR)

(21) Appl. No.: 18/257,500

(22) PCT Filed: Dec. 17, 2021
(86) PCT No.: PCT/NL2021/050773

§ 371 Date: Jun. 14, 2023

(30) Foreign Application Priority Data
Dec. 18, 2020 (EP) 20215349.0

(51) Int. Cl. A61K 38/22; A61K 47/61

(52) U.S. Cl. CPC A61K 38/22; A61K 47/61

(57) ABSTRACT

The present invention relates to the use of ACTH having the sequence of SEQ ID NO:1 in treating an adrenocorticotropic hormone (ACTH) responsive pediatric disorder, comprising administering twice daily to an individual in need thereof 0.5-2 mg/m² of body surface area of the ACTH. The present invention further relates to the use of ACTH having SEQ ID NO:1 in treating an adrenocorticotropic hormone (ACTH) responsive pediatric disorder, comprising administering twice daily to an individual in need thereof a treatment dose of between 0.5-2 mg/m² of body surface area of the ACTH for at least 7 days, optionally followed by the administration of a tapered dosage for an additional 14 days.

### FIELD OF THE INVENTION

The present invention relates to the novel and improved uses of adrenocorticotropic hormone (ACTH) in the treatment of an ACTH responsive pediatric disorder, such as infantile spasms. In particular, the invention relates to the use of novel and improved pharmaceutical formulations and administrations schemes. Specifically, the formulations comprise 0.5-2 mg/m² of ACTH having a specific sequence and administered twice daily.

### BACKGROUND OF THE INVENTION

Infantile spasms (IS), also West syndrome or infantile spasms syndrome, is a syndrome that develops in children younger than 2 years of age and is associated with frequent recurrent seizures (or spasms) and marked abnormal electroencephalograms (EEG) (interictal hypsarrhythmia and ictal voltage suppression). This rare disease is frequently associated with delayed development, permanent cognitive impairment, and the occurrence of other seizure types upon maturation. Death may also occur. The long-term prognosis of IS is poor as few patients mature to become neurodevelopmentally normal. Data suggest that early treatment of the spasms may improve long term neurologic prognosis.

The syndrome is considered to be catastrophic due to the frequent sequelae of global neurodevelopmental delay, significant intellectual disability, limited treatment options, and a poor prognosis (Wirrell 2016; Hancock et al 0213). In most cases, the initial age of onset is between 3 and 7 months, and over 90% of cases beginning before 12 months of life. The incidence of IS is 2 to 3.5 per 10,000 live births with a lifetime prevalence of 1.5 to 2 per 10,000 children. This translates to approximately 2000 to 2500 new cases per year in the United States (US). It is slightly more common in males, accounting for about 60% of cases, and a family history exists in 3% to 6% of cases. IS accounts for 25% to 30% of all cases of epilepsy affecting infants (National Organization for Rare Disorders—West syndrome).

Despite the development of new drugs and formulations in the recent decades, the outcomes of treatment of patients with infantile spasms remain unpredictable, with still many patients that do not benefit sufficiently from available treatment strategies. Considering the lack of a definitive treatment, there is a need for new therapies to treat an ACTH responsive pediatric disorder, including infantile spasms.

### SUMMARY OF THE INVENTION

The disclosure provides the following preferred embodiments. However, the invention is not limited to these embodiments.

In one aspect, the disclosure provides a method of treating an adrenocorticotropic hormone (ACTH) responsive pediatric disorder, comprising administering twice daily to an individual in need thereof between 0.5-2 mg/m2 of body surface area of ACTH having the sequence of SEQ ID NO:1. Preferably, the ACTH is administered twice daily for 14 days. Optionally, the ACTH is subsequently administered at a tapered dosage for an additional 14 days.

In a further aspect the disclosure provides adrenocorticotropic hormone (ACTH) having the sequence of SEQ ID NO:1 for use in the treatment of an ACTH responsive pediatric disorder by administering twice daily to an individual in need thereof 0.5-2 mg/m² of body surface area of said ACTH. Preferably, the ACTH is administered twice daily for 14 days. Optionally, the ACTH is subsequently administered at a tapered dosage for an additional 14 days.

In a further aspect, the disclosure provides a use of adrenocorticotropic hormone (ACTH) having the sequence of SEQ ID NO:1 in the manufacture of a medicament for the treatment of an ACTH responsive pediatric disorder by administering twice daily to an individual in need thereof 0.5-2 mg/m² of body surface area of said ACTH. Preferably, the ACTH is administered twice daily for 14 days. Optionally, the ACTH is subsequently administered at a tapered dosage for an additional 14 days.

In a further aspect, the disclosure provides a method of treating an adrenocorticotropic hormone (ACTH) responsive pediatric disorder, comprising administering twice daily to an individual in need thereof a treatment dose of between 0.5-2 mg/m² of body surface area of ACTH having the sequence of SEQ ID NO:1 for at least 7 days, optionally followed by the administration of a tapered dosage for an additional 14 days. Preferably, the treatment dose of between 0.5-2 mg/m² of ACTH is administered for 14 days.

In a further aspect, the disclosure provides adrenocorticotropic hormone (ACTH) having SEQ ID NO:1 for use in a method of treating an ACTH responsive pediatric disorder, by administering twice daily to an individual in need thereof a treatment dose of between 0.5-2 mg/m² of body surface area of ACTH having the sequence of SEQ ID NO:1 for at least 7 days, optionally followed with the administration of a tapered dosage for an additional 14 days. Preferably, the treatment dose of between 0.5-2 mg/m² of ACTH is administered for 14 days.

In a further aspect, the disclosure provides a use of adrenocorticotropic hormone (ACTH) having the sequence of SEQ ID NO:1 in the preparation of a medicament for treating an ACTH responsive pediatric disorder, by administering twice daily to an individual in need thereof a treatment dose of between 0.5-2 mg/m² of body surface area of ACTH having SEQ ID NO:1 for at least 7 days, optionally followed with the administration of a tapered dosage for an additional 14 days. Preferably, the treatment dose of between 0.5-2 mg/m² of ACTH is administered for 14 days.

In preferred embodiments, the ACTH responsive pediatric disorder is infantile spasms.

In preferred embodiments, the ACTH is administered twice daily at a dosage of 1 mg/m² per administration.

In preferred embodiments, at least 0.1 mg, 0.2 mg, at least 0.3 mg or at least 0.4 mg of ACTH is administered twice daily. Preferably, the daily dose (i.e. combined amount of two administrations) is between 0.2 and 1.6 mg, more preferably between 0.4 and 1.2 mg.

In preferred embodiments, the individual is a human child, preferably a human child of 2 years or younger. Preferably, the individual is a human child two months or older. In preferred embodiments, the individual is a human child having a body weight of at least 2 kg. In preferred embodiments, the individual is a human child having a body weight of at least 5 kg.

In preferred embodiments, the treatment with ACTH is started within 12 weeks after onset the ACTH responsive pediatric disorder, in particular infantile spasms, preferably within 12 weeks after onset of the first symptoms of the ACTH responsive pediatric disorder, in particular infantile spasms.

In preferred embodiments, the ACTH is synthetic or recombinant ACTH. Preferably, the ACTH is synthetic ACTH.

In preferred embodiments, the ACTH is provided in a pharmaceutical composition that does not comprise zinc. Preferably, the ACTH is provided in a pharmaceutical composition that does not comprise benzyl alcohol. Preferably, the ACTH is provided in a pharmaceutical composition comprising 0.3-0.8 mg/mL of ACTH, preferable comprising 0.5 mg/mL of ACTH.

### BRIEF DESCRIPTION OF THE DRAWINGS

FIG. 1 displays mean (±SD) plasma cortisol concentration by treatment versus time for the PD Analysis Set for all treatments.

FIG. 2 displays mean (±SD) plasma DHA concentration by treatment versus time, respectively, for the PD Analysis Set for all treatments.

FIG. 3 displays mean (±SD) plasma 17-hydroxypregnenolone concentration by treatment versus time, respectively, for the PD Analysis Set for all treatments.

### DETAILED DESCRIPTION OF THE DISCLOSED EMBODIMENTS

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 10% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Adrenocorticotropic hormone (ACTH) or corticotropin is the preferred first-line medical treatment in most IS patients (Glaze 2018-2) and has been used for approximately 6 decades in clinical practice to treat IS (Shields 2006). While the mechanism of action of ACTH for treating IS is poorly understood, it is hypothesized that ACTH may reduce neuronal excitability in IS by two mechanisms: (1) by inducing steroid release via activation of adrenal glucocorticoid receptors and (2) by a direct, steroid-independent action on melanocortin receptors (Gallo-Payet and Payet 2003).

H.P. Acthar® Gel is presently used in the US and several other countries in the treatment of IS. The recommended dose of ACTH as H.P. Acthar Gel is 75 U/m2, twice-daily, via intramuscular (IM) injection, for 14 days followed by a 14-day taper to discontinue. Outside of the US, two synthetic ACTH products are used to treat IS: Synacthen® Depot (in Canada, Europe, and elsewhere) and Acton Prolongatum™ (in India).

H.P. Acthar Gel (Baram et al. 1996; Hrachovy et al. 1986) is a biologically derived porcine ACTH. It is a naturally sourced complex mixture of adrenocorticotropic hormone analogs and other pituitary peptides. The Acthar Gel manufacturing process converts the initial porcine pituitary extract with low ACTH content into a mixture having modified porcine ACTH and other related peptide analogs solubilized in gelatin. As such, the formulation contains a mixture of ACTH-related substances, peptide impurities, and/or host-derived impurities. Amino acid sequence alignment between human ACTH and the ACTH sequence in H.P. Acthar Gel shows that the ACTH sequence in H.P. Acthar Gel differs by 3 amino acids compared to human ACTH (see Table 1). The ACTH sequence in the H.P. Acthar Gel contains Leu31 (as in the porcine sequence), but with additional substitutions at Asp25 and Gln30.

Synacthen® Depot is a suspension in which the ACTH is adsorbed onto an inorganic zinc complex matrix. The ACTH in Synacthen® Depot is a 24-mer peptide containing, from the N terminus, the first 24 of the 39 amino acids of natural human ACTH. The ACTH present in Acton Prolongatum is 39-mer synthetic porcine ACTH. During the manufacturing process of Acton Prolongatum in India, about 32 g of ACTH 1-39aa drug substance is used to prepare 60 L of the drug product labelled as 60 IU/mL. Consequently, about 0.5 mg of ACTH 1-39aa corresponds to 60 IU of ACTH 1-39aa in this product.

The present disclosure describes the development of a formulation of synthetic ACTH, a single-chain alpha amino acid polypeptide consisting of 39 amino acids. The full peptide sequence of synthetic ACTH is identical in sequence to porcine ACTH and differs from human ACTH by a single amino acid (see table 1). Given that the first 24 amino acid residues are identical between mammalian species and that the full peptide sequence of synthetic ACTH and human ACTH differs by a single amino acid, the disclosure provides that synthetic ACTH will exhibit the full corticosteroidogenic activity of natural human ACTH.

The present disclosure demonstrates the effectiveness of ACTH, as described herein, in a Phase 1, single-blind, active-controlled, single ascending dose (SAD) study comparing the pharmacodynamics (PD) of ACTH as described to H.P. Acthar Gel (target PD profile) and Cortrosyn (active control) in healthy adult subjects. Results from this Phase 1 study show that ACTH having SEQ ID NO:1 was generally safe and well tolerated at doses ranging from 0.15 mg to 0.75 mg. There were no deaths, treatment-emergent serious adverse events (SAEs), or withdrawals due to a treatment emergent adverse event (TEAE). All TEAEs were mild or moderate in severity and were resolved and did not cause any residual issues. Cortrosyn (the active control) and H.P. Acthar Gel (target PD profile) produced a rapid and marked increase in cortisol levels. Dose levels ≥0.25 and <0.5 mg of ACTH as described herein produce effects on cortisol and intermediates which are similar to those produced by a 25 U dose of H.P. Acthar Gel. The incidence of antibody formation following administration of the ACTH (defined as a minimum of 1 positive postdose result) was low and consistent with that observed for H.P. Acthar Gel. Accordingly, the ACTH described herein provides a clinically relevant treatment for infantile spasms.

TABLE 1
Amino acid sequences of ACTH peptides. Amino acids in bold denotes
amino acid differences in comparison to amino sequence of human ACTH.

| | Molecular | | |
|---|---|---|---|
| | Weight | | SEQ ID |
| ACTH Sequence | (g/mol) | Amino Acid Sequence | NO |
| synthetic porcine | 4567.23 | H-Ser-Tyr-Ser-Met-Glu-His-Phe- | SEQ ID |
| ACTH of the | | Arg-Trp-Gly-Lys-Pro-Val-Gly-Lys- | NO: 1 |
| present invention | | Lys-Arg-Arg-Pro-Val-Lys-Val-Tyr- | |
| | | Pro-Asn-Gly-Ala-Glu-Asp-Glu-Leu- | |
| | | Ala-Glu-Ala-Phe-Pro-Leu-Glu-Phe- | |
| | | OH | |
| Human ACTH | 4541.15 | H-Ser-Tyr-Ser-Met-Glu-His-Phe- | SEQ ID |
| | | Arg-Trp-Gly-Lys-Pro-Val-Gly-Lys- | NO: 2 |
| | | Lys-Arg-Arg-Pro-Val-Lys-Val-Tyr- | |
| | | Pro-Asn-Gly-Ala-Glu-Asp-Glu-Ser- | |
| | | Ala-Glu-Ala-Phe-Pro-Leu-Glu-Phe- | |
| | | OH | |
| H.P. Acthar Gel | 4567.23 | H-Ser-Tyr-Ser-Met-Glu-His-Phe- | SEQ ID |
| (biologically/ | | Arg-Trp-Gly-Lys-Pro-Val-Gly-Lys- | NO: 3 |
| naturally derived | | Lys-Arg-Arg-Pro-Val-Lys-Val-Tyr- | |
| porcine ACTH) | | Pro-Asp-Gly-Ala-Glu-Asp-Gln- | |
| | | Leu-Ala-Glu-Ala-Phe-Pro-Leu- | |
| | | Glu-Phe-OH | |
| Cortrosyn and | 2934 | H-Ser-Tyr-Ser-Met-Glu-His-Phe- | SEQ ID |
| Synachten ® | | Arg-Trp-Gly-Lys-Pro-Val-Gly-Lys- | NO: 5 |
| (24-mer of human | | Lys-Arg-Arg-Pro-Val-Lys-Val-Tyr- | |
| ACTH) | | Pro-OH | |

One aspect of the disclosure accordingly provides a method of treating an adrenocorticotropic hormone (ACTH) responsive pediatric disorder, comprising administering twice daily to an individual in need thereof 0.5-2 mg/m² of body surface area of ACTH having the sequence of SEQ ID NO:1. Also provided is ACTH having the sequence of SEQ ID NO:1 for use in a method of treating an ACTH responsive pediatric disorder by administering twice daily to an individual in need thereof 0.5-2 mg/m² of body surface area of said ACTH.

ACTH responsive pediatric disorders are known to one of skill in the art. Such disorders include epileptic spasms commonly referred to as infantile spasms. Infantile spasms (IS) is a syndrome that develops in children younger than 2 years of age and is associated with frequent recurrent seizures (or spasms) and marked abnormal electroencephalograms (EEG) (interictal hypsarrhythmia and ictal voltage suppression). As used herein, the “treatment of infantile spasms” can be used to refer to treatment of the specific seizure manifestations in patient suffering from the syndrome, but can also refer to treatment of the syndrome itself. In a preferred embodiment, the term “treatment of infantile spasms” refers to treatment of the syndrome infantile spasms. Hence, in preferred embodiments, the ACTH responsive pediatric disorder is infantile spasms.

As used herein the term “individual” preferably refers to a human, preferably a human child. In one embodiment, the individual is a human child 2 years or younger. In one preferred embodiment, the individual is a human child two months or older. The individual in need of treatment in accordance with the present disclosure is in particular suffering from an ACTH responsive pediatric disorder, in particular infantile spasms. In one preferred embodiment, the individual is a human child between two months and two years of age. In one preferred embodiment, the individual is a human child having a body weight of at least 3 kg. In one preferred embodiment, the individual is a human child having a body weight of at least 7 kg. On average, 7 kg is the body weight of boy of about 4.5 months and a girl of about 5.5 months.

The present disclosure is directed to the use of adrenocorticotropic hormone (ACTH) having the sequence of SEQ ID NO: 1. This ACTH is a peptide having 39 amino acid residues, corresponding to the sequence of porcine ACTH. The ACTH sequence used in accordance with the present disclosure is different at positions 25 and 30 from the sequence of the biologically/ naturally derived porcine ACTH in H.P. Acthar. Moreover, the ACTH used in accordance with the disclosure is comprised of intact ACTH polypeptide of 39 amino acids, rather than a mixture of peptide fragments.

In particular, the ACTH for administration or present in a formulation as described herein consists or consists essentially of ACTH having the sequence of SEQ ID NO: 1. In particular, the ACTH for administration does not include (significant) amounts of ACTH having SEQ ID NO:2 or SEQ ID NO:3. As used herein “does not include significant amounts” means that less than 1 wt % of the ACTH for administration or present in a formulation as described herein is ACTH having SEQ ID NO:2 or SEQ ID NO:3.

The ACTH can be administered as such, preferably formulated in a pharmaceutical composition. In one preferred embodiment, the ACTH is administered as a salt thereof for instance as the ACTH acetate salt.

The ACTH used in accordance with the present disclosure may be synthetic or recombinant ACTH. Preferably, the ACTH is synthetic ACTH. As used herein, “synthetic ACTH” means that the ACTH is chemically synthesized. An advantage of synthetic or chemically synthesized ACTH is that such ACTH is devoid of impurities, that may be present in a formulation comprising biologically derived or isolated ACTH, such as H.P. Acthar. Such formulations comprising biologically derived or isolated ACTH may contain a mixture of ACTH analogs, peptide impurities, and/or host-derived impurities. For instance, H.P. Acthar Gel can be considered as a naturally derived complex product where the contribution of the individual components to the safety and efficacy of the product has not been elucidated. Contrary, synthetic ACTH has a high purity and is associated with fewer side effects and thus improved safety, which is in particular of relevance for the highly vulnerable patient population of young children. In one preferred embodiment, the ACTH used in accordance with the present disclosure is provided in a pharmaceutical composition wherein the ACTH is the sole active ingredient.

The methods and uses of the present disclosure encompass the administration of the ACTH twice daily at a dose of 0.5-2 mg/m².

As used herein, “twice daily” means twice during each 24-hour period. This includes administration with equal or nearly equal time intervals, such as administration about every 12 hours, and administration at different time intervals between the individual administrations, such as administration with about 8 hours and about 16 hours intervals or with about 10 hours and about 14 hours intervals between two administrations. Preferably, the smallest time interval between two administrations in a 24-hour period is at least 4 hours, more preferably at least 5 hours, more preferably at least 6 hours, more preferably at least 7 hours, more preferably at least 8 hours, more preferably at least 9 hours, more preferably at least 10 hours. In one preferred embodiment, the time interval between each administration of twice daily administration regimen is between 8 and 16 hours. Preferably, the time interval between each administration is between 10 and 14 hours. More preferably, the time interval between each administration is about 12 hours. The amount of the two administrations of ACTH during a 24-hour period is such that in total a dose of 1-4 mg/m² of body surface area is achieved, whereby the amounts of both administrations may the approximately equal or different, as long as they are each 0.5-2 mg/m² of body surface area. Preferably, the amount of ACTH that is administered with each individual administration during each 24-hour period is the approximately the same.

As used herein “daily” administration means administration of one dose per 24 hours. In one preferred embodiment, the time interval between each individual administration is between 12 and 36 hours, more preferably between 20 and 28 hours. In one preferred embodiment, the time interval between each administration is about 24 hours.

As used herein administration “once every two days” means administration of one dose per 48 hours. In one preferred embodiment, the time interval between each individual administration is between 36 and 60 hours, more preferably between 42 and 56 hours. Preferably, the time interval between each administration is about 48 hours. A dose administered once every two days for 5 days following daily administration preferably means that the dose is administered at day 1, 3 and 5 following the final dose of the daily administration.

The dosage of each administration in accordance with the present disclosure of twice daily administration between 0.5 and 2 mg/m 2 of body surface area is based on the weight of the ACTH in total. Hence, a total amount of between 1 and 4 mg/m² ACTH is administered daily. In one preferred embodiment 0.5-1.5 mg/m² of the ACTH is administered twice daily in accordance with the present disclosure. Hence, a total amount of between 1 and 3 mg/m² is administered daily. In one preferred embodiment 0.8-1.2 mg/m² of the ACTH is administered twice daily in accordance with the present disclosure. Hence, a total amount of between 1.6 and 2.4 mg/m² is administered daily. Preferably, about 1 mg/m 2 of the ACTH is administered twice daily in accordance with the present disclosure. Hence, preferably, a total amount of between about 2 mg/m² is administered daily.

The dosages described herein thus differ from the recommended dosages for Acton Prolongatum, which are a “fixed” dosage; namely, 20 units. In addition, the resulting amounts of ACTH administered are higher than the fixed dosage prescribed by Acton Prolongatum. The disclosure provides that the dosing regimens of the invention described herein are both safe and effective.

In some embodiments, the individual has a body surface area of at least 0.2 m². In some embodiments, the individual has a body surface area of at least 0.3 m². Preferably, the total daily dosage of ACTH (calculated based on BSA) is at least 0.2 mg, at least 0.3 mg, at least 0.4 mg, or at least 0.6 mg. In some embodiments, the total daily dosage is between 0.2 mg to 1.6 mg.

As used herein “mg/m²” refers to the amount of mg per m² of body surface area (BSA) of the individual the ACTH is administered to. A number of different formulas can be used to calculate the body surface area giving slightly different results. The most commonly used formula is that of Mosteller, published in 1987 (Mosteller R D. Simplified calculation of body-surface area. N Engl J Med 1987; 317:1098.). According to Mosteller's calculation, the body surface area=the square root of product of the weight in kg times the height in cm divided by 3600 or BSA (m²)=([Height(cm)×Weight(kg)]/3600)^{1/2}. In a preferred embodiment, the dosage as used herein in mg/m² is based on the body surface area as calculated by the formula BSA (m²)=([Height(cm)×Weight(kg)]/3600)^{1/2}. Accordingly, the exact dose per administration and dosage per day depends on the age, weight and length of the individual.

Table 2 provides an average of BSA for children of indicated age and weight, and the average dosage in mg and daily dosage in mg of an exemplary dosage regimen of 1 mg/m² twice daily.

TABLE 2
Average BSA for children and dosing of ACTH.

| Body weight (kg) | | Surface area | Dosage per | Dosage per |
|---|---|---|---|---|
| Approximate age- boy | | (m2) | administration (mg) | day (mg) |
| 2 | | 0.16 | 0.16 | 0.32 |
| | | 0.2 | 0.2 | 0.4 |
| 5 | (2 m) | 0.3 | 0.3 | 0.6 |
| | | 0.32 | 0.32 | 0.64 |
| 7 | (4 m) | 0.38 | 0.38 | 0.76 |
| | | 0.4 | 0.4 | 0.8 |
| 8 | (7 m) | 0.42 | 0.42 | 0.84 |
| 10 | (12 m) | 0.49 | 0.49 | 0.98 |
| 14 | (3 y) | 0.62 | 0.62 | 1.24 |
| 19 | (5 y) | 0.77 | 0.77 | 1.54 |

The ACTH may be administered to the individual, preferably human child, continuously, short term or intermittently. In one preferred embodiment, the dosage of between 0.5 and 2 mg/m², preferably 0.5-1.5 mg/m², more preferably 0.8-1.2 mg/m², such as about 1 mg/m², is administered twice daily for at least 7 days, more preferably twice daily for at least 10 days, more preferably twice daily for about 14 days. Preferably, said ACTH is administered twice daily for 7-21 days, more preferably 10-17 days, more preferably for about 14 days.

It is further preferred that the ACTH is administered in a dosing schedule comprising at least two phases, a first phase wherein a treatment dosage is administered at twice daily frequency, and a second phase wherein a second dosage is administered. In one preferred embodiment, the second dose is a tapered dose. As used herein the term “tapered dosage” refers to administration of multiple doses in amounts that decrease over time, i.e. whereby each dose is equal to or lower than the preceding dose, and equal doses are administered for at most 5 days with the exception of the final dose. The final dose can be provided continuously, short term for e.g. at most 5 or 10 days or intermittently.

The disclosure therefore also provides a method of treating adrenocorticotropic hormone (ACTH) responsive pediatric disorder, comprising administering twice daily to an individual in need thereof between 0.5-2 mg/m² of body surface area of ACTH having the sequence of SEQ ID NO:1 for at least 7 days, preferably for 7-14 days, more preferably for about 14 days, followed by administration of a tapered dose of the ACTH. Also provided is ACTH having the sequence of SEQ ID NO:1 for use in a method of treating ACTH responsive pediatric disorder, comprising administering twice daily to an individual in need thereof between 0.5-2 mg/m² of body surface area of the ACTH for at least 7 days, preferably for 7-14 days, more preferably for about 14 days, followed by administration of a tapered dose of the ACTH. In one preferred embodiment, the ACTH is administered twice daily at a dose of between 0.5 and 2 mg/m², preferably 0.5-1.5 mg/m², more preferably 0.8-1.2 mg/m², such as about 1 mg/m², for 7 to 21 days in accordance with the disclosure, followed by administration of said ACTH at a tapered dose. Said tapered dose preferably is administered once or twice daily or once every two days, more preferably once daily or once every two days.

In one preferred embodiment, each individual dose of the tapered dosage is between 0.1 and 0.5 mg/m 2 of body surface area of ACTH having the sequence of SEQ ID NO:1. Hence, in one preferred embodiment the individual is administered twice daily a dose of between 0.5 and 2 mg/m² of body surface area, preferably 0.5-1.5 mg/m², more preferably 0.8-1.2 mg/m², such as about 1 mg/m², for about 7 to 21 days, followed by daily administration of said ACTH at a tapered dose of between 0.1 and 0.5 mg/m² of body surface area for at least 7 days, optionally followed by administration of said ACTH at a dose of 0.1 mg/m² every other day for at least 3 days.

In one preferred embodiment the individual is administered twice daily a dose of about 1 mg/m² of body surface area for 14 days, followed by daily administration of said ACTH at a dose of about 0.4 mg/m² once daily for 3 days, followed by daily administration of said ACTH at a dose of about 0.2 mg/m 2 once daily for 3 days, followed by daily administration of said ACTH at a dose of about 0.1 mg/m² once daily for 3 days, followed by daily administration of said ACTH at a dose of about 0.1 mg/m² once every two days for 5 days, i.e. administration on day 1, 3 and 5 of these 5 days.

It is preferred that treatment in accordance with the inventions starts as soon as possible. The treatment with ACTH in accordance with the invention is therefore preferably started within 12 weeks after onset the ACTH responsive pediatric disorder, in particular infantile spasms, preferably within 12 weeks after onset of the first symptoms of the ACTH responsive pediatric disorder, in particular infantile spasms. More preferably, the treatment with ACTH is therefore preferably started within 12 weeks after onset the ACTH responsive pediatric disorder, in particular infantile spasms, preferably within 12 weeks after onset of the first symptoms of the ACTH responsive pediatric disorder, in particular infantile spasms.

The ACTH can be administered via any suitable route in the methods and uses of the present disclosure. Preferably the ACTH is administered by injection, including intravenous, intramuscular and subcutaneous injection. In one preferred embodiment, the ACTH is administered intramuscularly, e.g. in the deltoid muscle (in the upper arm), the vastus lateralis muscle (of the thigh), the ventrogluteal muscle (of the hip) or the dorsogluteal muscle (of the buttock). A skilled person is well capable of selecting a suitable site for intramuscular injection in accordance with the present disclosure.

Preferably, the ACTH used in accordance with the present disclosure is formulated in a pharmaceutical composition. Methods of formulation of pharmaceutical compositions are well known in the art. The pharmaceutical composition according to the invention is preferably suitable for human use. The composition may also include pharmaceutically acceptable additives such as carriers, diluents and/or excipients. By “pharmaceutically acceptable” it is meant that the carrier, diluent or excipient must be compatible with the other ingredients of the formulation and not deleterious, e.g. toxic, to the recipient thereof. In general, any pharmaceutically suitable additive which does not interfere with the function of the active compounds can be used.

A pharmaceutical composition comprising the ACTH having a sequence of SEQ ID NO:1 and used in accordance with the disclosure is preferably formulated for parenteral administration, more preferably injection. Subcutaneous, intramuscular and intravenous injection are all suitable routes for dosing. In one preferred embodiment, the pharmaceutical composition is formulated for intramuscular administration.

Pharmaceutical compositions for administration by injection, such as intramuscular, intravenous or subcutaneous administration, may for example be solutions of the ACTH as described herein in sterile isotonic aqueous solution. Where necessary, the compositions may include for instance solubilizing agents, stabilizing agents and/or a local anesthetic to ease the pain at the site of the injection. In one preferred embodiment, the pharmaceutical composition is a sterile formulation. In one preferred embodiment, the ACTH is provided in a pharmaceutical composition that does not comprise zinc. In one preferred embodiment, the ACTH is provided in a pharmaceutical composition that does not comprise benzyl alcohol. Benzyl alcohol has been associated with the risk of severe side effects including respiratory failure, vasodilation and hypotension in young children.

In one preferred embodiment, the formulation contains carboxymethylcellulose. In one preferred embodiment, the ACTH in the formulation is bound to carboxymethylcellulose. Carboxymethylcellulose may protect the ACTH against proteolytic degradation and/or increase stability. It further provides viscosity to the formulation.

Methods for formulation of pharmaceutical compositions, including those formulated for intramuscular administration, are well known in the art. In one preferred embodiment, the ACTH is provided in a pharmaceutical composition comprising between 0.3 and 0.8 mg/mL of ACTH. In one preferred embodiment, the ACTH is provided in a pharmaceutical composition comprising about 0.5 mg/mL of ACTH.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1 Phase 1, Active-Controlled, Single Ascending Dose Study: the Pharmacodynamics of AMZ002

### Methods and Materials

This was a Phase 1, single-blind, active-controlled, single ascending dose study to compare the PD effects of the investigational drug AMZ002 with those of H.P. Acthar Gel and Cortrosyn in healthy adult subjects. AMZ002 is a sterile solution for IM injection, formulated to contain 0.5 mg/mL ACTH having the sequence Ser-Tyr-Ser-Met-Glu-His-Phe-Arg-Trp-Gly-Lys-Pro-Val-Gly-Lys-Lys-Arg-Arg-Pro-Val-Lys-Val-Tyr-Pro-Asn-Gly-Ala-Glu-Asp-Glu-Leu-Ala-Glu-Ala-Phe-Pro-Leu-Glu- Phe, 50 mg/mL dextrose, 10 mg/mL sodium carboxymethylcellulose, and 5 mg/mL phenol (pH 6.4). The concentration of 0.5 mg/mL was chosen to reduce possible errors resulting from the preparation of small dose volumes (e.g., less than 0.4 ml).

Subjects who met all of the inclusion criteria and none of the exclusion criteria were enrolled in the study. Safety and tolerability were evaluated throughout the study, including all adverse events (AEs); physical examination findings; vital sign measurements; 12-lead electrocardiogram (ECG) findings; standard clinical laboratory measurements (eg, serum chemistry, hematology, and urinalysis); and injection site evaluations. Immunogenicity, as measured by antidrug antibodies (ADA) were evaluated. PD effects were assessed based on levels of cortisol and an adrenal panel of intermediates (dehydroepiandrosterone [DHA], 17-hydroxypregnenolone, and 17 hydroxyprogesterone). Blood samples for measurement of syn-ACTH concentrations were also collected (AMZ002 cohorts only).

The study consisted of 8 cohorts, with 8 subjects per cohort (each a single dose): Cortrosyn 0.25 mg; H.P. Acthar Gel 25 U; or, in an ascending dose fashion, AMZ002 0.15, 0.20, 0.25, 0.30, 0.50, or 0.75 mg. The investigational product (AMZ002) and active comparators (Cortrosyn and H.P. Acthar Gel) were administered via intramuscular (IM) injection to the upper arm. The dose of Cortrosyn (0.25 mg by IM administration), the active control, was intended to produce maximal adrenal stimulation. This is the standard dose for adults and children ≥2 years of age recommended in the 2015 Endocrine Society Clinical Practice Guidelines for diagnostic testing of adrenal insufficiency (same dose whether Cortrosyn is administered intravenously or IM). Clinical pharmacology studies for Cortrosyn indicate that 0.25 mg Cortrosyn stimulates the adrenal cortex to the same extent as 25 U of purified natural ACTH (Cortorsyn® (cosyntropin) for Injection [package insert]. Rancho Cucamonga, CA. Amphastar Pharmaceuticals, Inc. November 2016). Since 25 U of natural ACTH was expected to elicit maximal adrenal response, it was considered the most sensitive dose to determine a pharmacodynamically equivalent dose of AMZ002 in this study. Therefore, this study included a cohort of subjects who received a single dose of 25 U H.P. Acthar Gel (representing the target PD for the investigational drug product in this study).

On Day-1, a single standard dose of dexamethasone (1 mg) was administered orally at approximately midnight (8 hours prior to study drug administration the following day) to suppress endogenous adrenocorticotropic hormone (ACTH) production and remove this as a confounding variable in the measurements. The 1 mg dose was expected to produce adequate suppression of endogenous ACTH levels in healthy subjects to allow the study objectives to be met. Study drug was administered at 8:00 AM (±1 hour) on Day 1.

Blood samples were collected prior to dosing on Day 1; at 5, 15, 30, and 45 minutes postdose; and at 1, 1.5, 2, 2.5, 3, 4, 5, 6, 8, 10, 12, 24, 30, 36, and 48 hours postdose.

For each PD (pharmacodynamics) analyte (plasma cortisol and an adrenal panel of intermediates [DHA, 17-hydroxypregnenolone, and 17-hydroxyprogesterone]), the following parameters were calculated with the actual time points using a noncompartmental method as appropriate:
Maximum observed PD measurement (Eₘₐₓ);
Time to reach maximum observed PD measurement (T_{Emax}); and
Area under the PD effect-time curve from time 0 to the last quantifiable PD measurement (AUEC₀₋ₜ).

The PD parameters using baseline-corrected PD measurements were calculated similarly, if applicable. Baseline was defined as the measurement at predose for each PD analyte. The following parameters were also calculated using baseline-corrected PD measurements:
Area under the PD effect-time curve that was above baseline (AUEC_{above B}); and
Total time that response was≥baseline (Time_{above B}).

Actual time points were used in the PD variable calculations. A linear trapezoidal/linear interpolation method was used in the calculation of area under the plasma concentration-time curves (AUCs).

The following plasma PK (pharmacokinetics) parameters of syn-ACTH were calculated from individual syn-ACTH concentration data for each cohort:
Maximum plasma concentration (Cₘₐₓ) if the maximum value occurred at more than 1 time point, Cₘₐₓ was defined as the first value;
Time to Cₘₐₓ (Tₘₐₓ);
Terminal elimination constant rate (λ_{z});
Terminal elimination half-life (t_{1/2}), calculated as ln(2)/λ_{z};
AUC from time 0 to the last quantifiable plasma concentration (Cₗₐₛₜ) (AUCₗₐₛₜ);
AUC from time 0 to infinity (AUC_{inf});

Proportion of AUC_{inf} due to extrapolation (%)(AUCₑₓₜᵣ), calculated as 100×(Cₗₐₛₜ/λ_{z})/AUC_{inf};
Apparent clearance (CL/F); calculated as Dose/AUC_{inf}; and
Apparent volume of distribution (V_{z}/F), calculated as Dose/[λ_{z}×AUC_{inf}].

Blood samples for evaluation of immunogenicity as measured by ADA were obtained at the Treatment Visit (Day 1) (predose), prior to discharge (Day 3), the Follow-Up Visit (Day 8 [+2 days])/Early Termination Visit, and subsequent visits for collection of ADA (Day 15 [+2 days] and Day 29 [+2 days]).

Safety parameters included all AEs; physical examination findings; vital sign measurements; 12-lead ECG findings; standard clinical laboratory measurements (eg, serum chemistry, hematology, and urinalysis); and injection site evaluations.

### Results

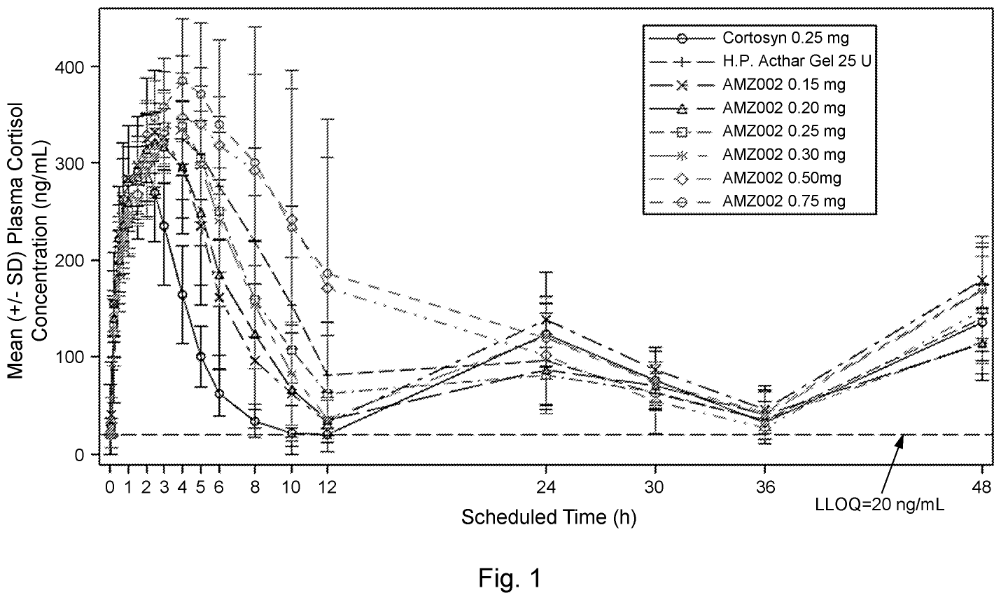

Cortrosyn and H.P. Acthar Gel produced a rapid and marked increase in cortisol levels (see FIG. 1).

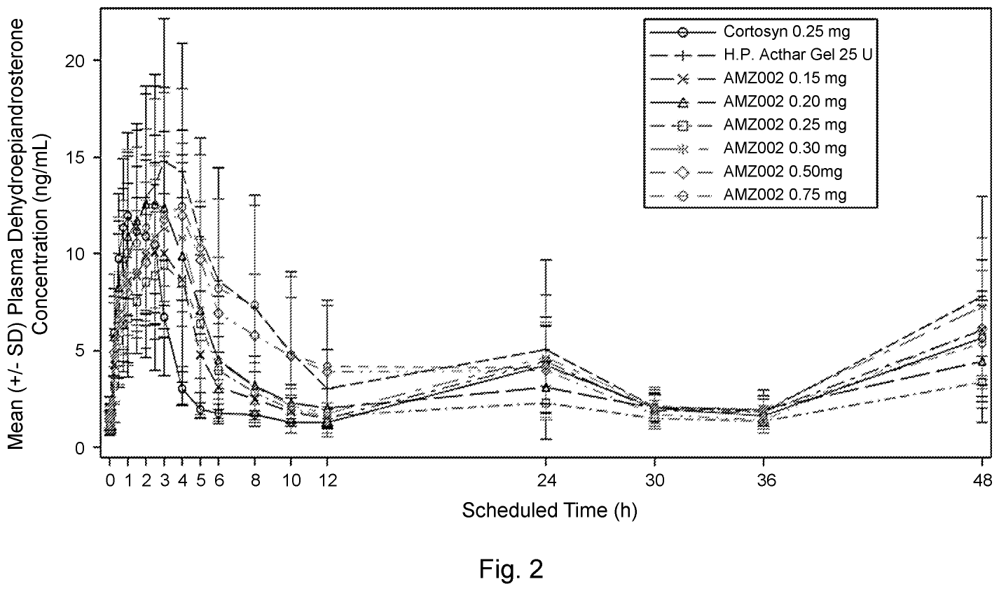

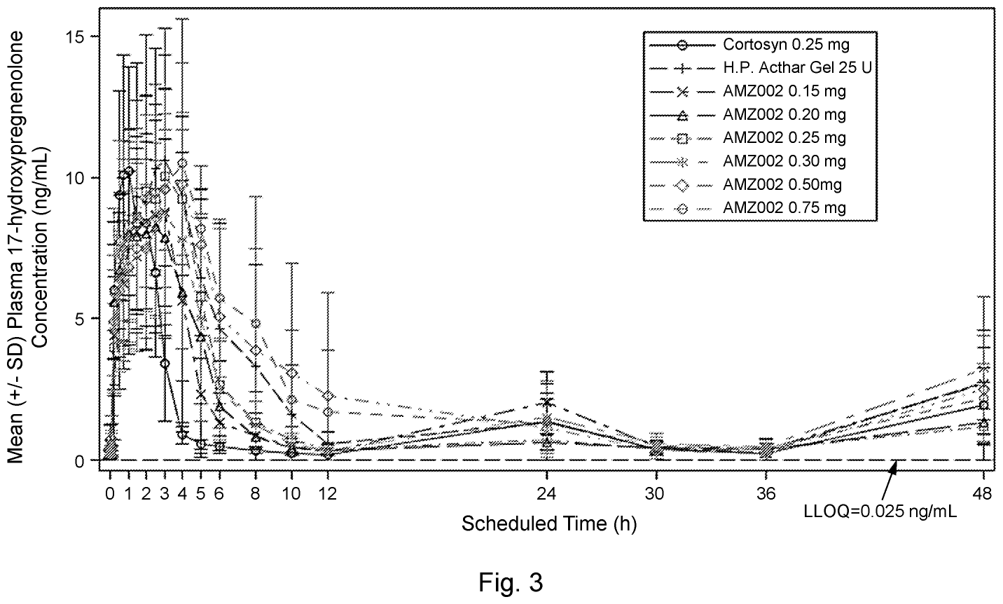

Doses ranging from 0.15 to 0.75 mg of AMZ002 bracket the H.P. Acthar Gel cortisol response with the lowest levels of AMZ002 producing a maximal effect that is less sustained as compared to 25 U H.P. Acthar Gel and the highest levels producing slightly greater and more sustained effects on cortisol as compared to 25 U H.P Acthar Gel (see FIG. 1). Similar findings were observed for the intermediates DHA (FIGS. 2) and 17-hydroxypregnenolone (FIG. 3), with lower levels of AMZ002 generally producing a maximal effect that is less sustained as compared to higher levels.

Dose levels ≥0.25 and ≤0.5 mg AMZ002 are generally appropriate to produce effects on cortisol and intermediates that are similar to those produced by a 25 U dose of H.P. Acthar Gel.

Following administration of a single dose, AMZ002 was rapidly absorbed, with mean peak plasma concentrations of AMZ002 obtained within 2 hours after dosing (median Tmax values ranged from approximately 0.08 to 2.00 hours). Plasma concentrations of AMZ002 declined from peak rapidly with mean t½ values ranged from approximately 0.911 to 2.594 hours. Exposures to AMZ002 generally increased with increase in dose, but in a greater than proportional manner.

All treatment emergent adverse events (TEAEs) were resolved and did not cause any residual issues. Three AEs required treatment with concomitant medications for resolution, but were mild in severity. There were no deaths, treatment-emergent serious adverse events, or withdrawals due to a TEAE.

AMZ002 was safe and well tolerated at all doses studied.

### Conclusions

Dose levels ≥0.25 and ≤0.5 mg AMZ002 are generally appropriate to produce effects on cortisol and intermediates that are similar to those produced by a 25 U dose of H.P. Acthar Gel.

### REFERENCES

Baram T Z, Mitchell W G, Tournay A, et al. High-dose corticotropin (ACTH) versus prednisone for infantile spasms: a prospective, randomized, blinded study. Pediatrics. 1996; 97(3):375-379.
Gallo-Payet N, Payet M D. Mechanism of action of ACTH: beyond cAMP. Microsc Res Tech. 2003; 61(3):275-287.
Hancock EC, Osborne J P, Edwards S W. Treatment of infantile spasms. Cochrane Database Syst Rev. 2013; (6):CD001770.
Hrachovy R A, Frost J D Jr, Kellaway P, et al. Double-blind study of ACTH vs prednisone therapy in infantile spasms. J Pediatr. 1983; 103(4):641-645.
Shields W D. Infantile spasms: little seizures, BIG consequences. Epilepsy Curr. 2006; 6(3):63-69.
Wirrell E. Infantile, childhood, and adolescent epilepsies. Continuum (Minneap Minn). 2016; 22(1):60-93.

1. A method of treating an adrenocorticotropic hormone (ACTH) responsive pediatric disorder, comprising administering twice daily to an individual in need thereof 0.5-2 mg/m² of body surface area of ACTH having the sequence of SEQ ID NO:1, wherein the ACTH is provided in a pharmaceutical composition wherein the ACTH is the sole active ingredient.

2. (canceled)

3. The method of claim 1, wherein the ACTH is administered at a dosage of 1 mg/m² of body surface area twice daily.

4. The method of claim 1, wherein at least 0.1 mg of ACTH is administered twice daily, wherein at least 0.2 mg of ACTH is administered twice daily or wherein at least 0.3 mg of ACTH is administered twice daily.

5. The method of claim 1, wherein the individual is a human child 2 years or younger.

6. The method of claim 1, wherein the individual is a human child two months or older.

7. The method of claim 1, wherein the individual is a human child having a body weight of at least 2 kg or wherein the individual is a human child having a body weight of at least 5 kg.

8. The method of claim 1, wherein the ACTH is synthetic ACTH.

9. The method of claim 1, wherein the ACTH is provided in a pharmaceutical composition that does not comprise zinc and/or that does not comprise benzyl alcohol.

10. The method of claim 1, wherein the ACTH is provided in a pharmaceutical composition comprising between 0.3-0.8 mg/mL of ACTH.

11. The method of claim 1, wherein the ACTH is administered intramuscularly.

12. The method of claim 1, wherein the ACTH is administered twice daily for 14 days.

13. The method of claim 1, wherein the formulation contains carboxymethylcellulose.

14. The method of claim 1, wherein the ACTH in the formulation is bound to carboxymethylcellulose.

15. A method of treating an adrenocorticotropic hormone (ACTH) responsive pediatric disorder, comprising administering twice daily to an individual in need thereof a treatment dose of between 0.5-2 mg/m² of body surface area of ACTH having SEQ ID NO:1 for at least 7 days, optionally followed by the administration of a tapered dosage for an additional 14 days, wherein the ACTH is provided in a pharmaceutical composition wherein the ACTH is the sole active ingredient.

16. (canceled)

17. The method of claim 1 wherein the ACTH responsive pediatric disorder is infantile spasms.

18. The method of claim 1, wherein the ACTH is provided in a pharmaceutical composition comprising 0.5 mg/mL of ACTH.

19. The method of claim 12, wherein the ACTH is administered at a tapered dosage for an additional 14 days.


SEQUENCE LISTING (5 sequences)

SEQ ID NO: 1 - protein, 39 aa, Artificial Sequence
  synthetic porcine ACTH of the present invention
SYSMEHFRWGKPVGKKRRPVKVYPNGAEDELAEAFPLEF

SEQ ID NO: 2 - protein, 39 aa, Homo sapiens
SYSMEHFRWGKPVGKKRRPVKVYPNGAEDESAEAFPLEF

SEQ ID NO: 3 - protein, 39 aa, Artificial Sequence
  H.P. Acthar Gel (biologically/ naturally derived porcine ACTH)
SYSMEHFRWGKPVGKKRRPVKVYPDGAEDQLAEAFPLEF

SEQ ID NO: 4 - protein, 10 aa, Artificial Sequence
  placeholder sequence
AAAAAAAAAA

SEQ ID NO: 5 - protein, 24 aa, Artificial Sequence
  Cortrosyn and Synachten (24-mer of human ACTH)
SYSMEHFRWGKPVGKKRRPVKVYP